(19) United States
(12) Patent Application Publication
(10) Pub. No.: US 2021/0007423 A1
(43) Pub. Date: Jan. 14, 2021

(54) WEBBING STRAP DEVICE OF ADJUSTABLE LENGTH AND FUNCTIONAL DEVICE HAVING A WEBBING STRAP DEVICE

(71) Applicant: UVEX ARBEITSSCHUTZ GMBH, Fürth (DE)

(72) Inventors: Bernd NEUBAUER, Nürnberg (DE); Daniel SCHEMM, Langenzenn (DE); Uwe FRIEDLEIN, Emskirchen (DE)

(21) Appl. No.: 16/980,640

(22) PCT Filed: Jan. 30, 2019
(86) PCT No.: PCT/EP2019/052268

§ 371 Date: Sep. 14, 2020

(30) Foreign Application Priority Data
Mar. 15, 2018 (DE) 10 2018 204 007.9

(51) Int. Cl. A41D 13/11; A44B 11/04; A61F 9/02

(52) U.S. Cl. CPC A41D 13/1161; A61F 9/027; A44B 11/04

(57) ABSTRACT

A webbing strap device of adjustable length include an adjustment strap anchor, an adjustment loop anchor with an adjustment loop eyelet, a ladder-shaped sliding body with a first frame crossbar, a second frame crossbar spaced from the first frame crossbar and at least two intermediate crossbars arranged between the first frame crossbar and the second frame crossbar, and a webbing strap attached to the first frame crossbar and to the adjustment strap anchor. The webbing strap extends through the slider between the at least two intermediate crossbars to form an adjustment loop. In the region of the adjustment loop, the webbing strap extends through the adjustment loop eyelet.

This application claims the priority of German Patent Application Serial No. DE 10 2018 204 007.9, filed on Mar. 15, 2018, pursuant to 35 U.S.C. 119(a)-(d), the content of which is incorporated herein by reference in its entirety as if fully set forth herein.

### FIELD OF THE INVENTION

The invention concerns a webbing strap device of adjustable length. The invention furthermore concerns a functional device having such a webbing strap device.

### BACKGROUND OF THE INVENTION

Webbing strap devices are generally known from the prior art. These normally comprise an adjustment strap anchor, an adjustment loop anchor, a ladder-like slider with a first frame crossbar, a second frame crossbar spaced from the first frame crossbar, and a single intermediate crossbar arranged between the first frame crossbar and the second frame crossbar, and a webbing strap attached to the intermediate crossbar and to the adjustment strap anchor. The webbing strap extends through the slider between the first frame crossbar and the intermediate crossbar to form an adjustment loop. The webbing strap passes twice through the slider between the first frame crossbar and the intermediate crossbar. For automatic production of the webbing strap device, passing the webbing strap through the slider repeatedly is a hindrance. The threading process is complex and not robust in operation. Such a webbing strap device is known for example from US D477,347 S.

Alternative embodiments of webbing strap devices are known for example from DE 10 2012 014 693 A1 U.S. Pat. No. 2,022,483 A and EP 1 389 434 A1.

### SUMMARY OF THE INVENTION

The invention is based on the object of providing an improved webbing strap device. The webbing strap device can be produced particularly economically and efficiently in a robust process.

This object is achieved by a webbing strap device of adjustable length, including an adjustment strap anchor, an adjustment loop anchor with an adjustment loop eyelet, a ladder-shaped slider with a first frame crossbar, a second frame crossbar spaced from the first frame crossbar and at least two intermediate crossbars arranged between the first frame crossbar and the second frame crossbar, and a webbing strap attached to the first frame crossbar and to the adjustment strap anchor, wherein the webbing strap extends through the slider between the at least two intermediate crossbars to form an adjustment loop, and wherein in the region of the adjustment loop, the webbing strap extends through the adjustment loop eyelet. The core of the invention is that the slider has at least two intermediate crossbars arranged between the first frame crossbar and the second frame crossbar, and that the webbing strap is guided through the slider between the at least two intermediate crossbars in order to form the adjustment loop. The webbing strap is here fixed to the first frame crossbar and to the adjustment strap anchor. Thus there is no need for the webbing strap to pass repeatedly through the slider between the first frame crossbar and the at least two intermediate crossbars. Advantageously, this ensures that the webbing strap device according to the invention can be produced automatically and hence particularly economically.

Preferably, both ends of the first frame crossbar and/or the second frame crossbar are each connected to a frame strut. The two frame struts together with the first frame crossbar and the second frame crossbar may form a closed slider frame. Preferably, the at least two intermediate crossbars are also connected to a respective one of the two frame struts at both ends. The at least two intermediate crossbars may in particular be connected to one of the frame struts only on one side. In particular, at least part of the frame crossbars, the at least two intermediate crossbars and the frame struts may have an interruption for passage of the webbing strap. Preferably, the first frame crossbar and/or the second frame crossbar and/or the at least two intermediate crossbars are oriented parallel to each other. Thus the webbing strap can be guided through the slider particularly evenly, and tilting of the slider can be reliably prevented.

The slider has a first strap opening for passage of the webbing strap adjacent to the first frame crossbar and between this and the first of the at least two intermediate crossbars. The slider has a second strap opening for passage of the webbing strap adjoining the second frame crossbar and between this and a second of the at least two intermediate crossbars. The slider has at least one further, namely middle strap opening between each pair of mutually adjacent intermediate crossbars. The slider may have at least three, in particular at least four, in particular at least five intermediate crossbars.

Preferably, the slider has precisely two intermediate crossbars. The slider thus preferably has in total precisely three strap openings. Particularly preferably, the webbing strap is guided through the first strap opening and through the second strap opening and through the middle strap opening.

The webbing strap may be attached to the first frame crossbar and/or to the adjustment strap anchor by force fit, in particular in the form of a clamp connection and/or a screw connection, and/or by form fit, in particular in the form of a bead connection and/or a pin connection and/or a stitching, and/or by substance bonding, in particular by gluing and/or welding. The webbing strap is thus connected to the first frame crossbar and/or to the adjustment strap anchor in a particularly fixed and reliable fashion.

The webbing strap may however, in particular, also be formed from several pieces. Preferably, the webbing strap is formed from two pieces, wherein a closing means is arranged between a first part and a second part of the webbing strap. The closing means may comprise a catch connection for reversible connection of the first part and second part of the webbing strap. Advantageously, this achieves that the webbing strap device can be arranged in an open position and/or a closed position.

The webbing strap may comprise a woven and/or a knitted and/or a worked fabric. Preferably, the webbing strap consists completely of a woven and/or knitted and/or worked textile material.

Preferably, the closing means is configured such that it can be guided through at least one, in particular all of the strap openings. The webbing strap can thus be exchanged particularly easily.

Preferably, the webbing strap runs through all strap openings, in particular the first strap opening and the second strap opening and the at least one middle strap opening. The friction between the webbing strap and the slider is thus increased, and undesirable displacement of the webbing strap relative to the slider is reliably prevented.

Preferably, the webbing strap has no loose end. A loose end means an end which can vary in its extent with the length of the webbing strap device. Preferably, both ends of the webbing strap are attached to the adjustment strap anchor and/or to the adjustment loop anchor and/or to the slider, and hence there are no loose ends. By avoiding such loose ends, the wearing comfort of the webbing strap device is additionally increased.

A webbing strap device, wherein the webbing strap extends through the slider adjacent to the second frame crossbar, between this and the at least two intermediate crossbars, can be adjusted and set particularly reliably. Because the webbing strap extends both through the at least one middle strap opening and also through the second strap opening, the friction between the webbing strap and the slider is increased. Unintentional displacement of the webbing strap relative to the slider may thus be prevented. Preferably, a length of the respective strap opening corresponds approximately to a width of the webbing strap. A width of the first strap opening and/or the second strap opening and/or the at least one middle strap opening is preferably at most 6 mm, in particular at most 4 mm, in particular at most 3 mm, in particular at most 2 mm. A depth of the first strap opening and/or the second strap opening and/or the at least one middle strap opening is preferably at least 1 mm, in particular at least 2 mm, in particular at least 4 mm, in particular at least 6 mm. This may securely prevent an unintentional displacement of the webbing strap relative to the slider.

A webbing strap device, wherein adjacent passages of the webbing strap through the slider are separated from each other by means of one of the at least two intermediate crossbars, can be produced automatically and economically. The term “passage” means an extent of the webbing strap through the slider, in particular through one of the strap openings. Preferably, mutually adjacent passages are separated from each other by at least one of the at least two intermediate crossbars. Because the webbing strap passes through each of the strap openings in particular at most and preferably precisely once, the webbing strap device can be produced reliably and robustly in an automated process.

A webbing strap device, wherein the webbing strap runs around the at least two intermediate crossbars only on one side, can be produced automatically and economically. Because the webbing strap runs around the at least two intermediate crossbars only on one side, the webbing strap can be guided through the slider, in particular the strap openings, particularly easily.

A webbing strap device, wherein the webbing strap is formed as one piece, can be produced particularly economically and has a high wearing comfort. A one-piece design means that the webbing strap consists of a single strap, in particular without joining seam. The one-piece webbing strap in particular has no adhesive seam and/or no stitching via which it is assembled from several part pieces. Because such joining seams are avoided, the webbing strap device has a particularly high wearing comfort.

A webbing strap device, wherein the webbing strap is formed so as to be elastic and has a stretch elongation of at least 10%, guarantees a firm seat and has a high wearing comfort. The stretch elongation of the webbing strap is preferably at least 10%, in particular at least 20%, in particular at least 40%, in particular at least 60%, in particular at least 100%. For this, the webbing strap may comprise an elastic, in particular a rubber elastic material. The webbing strap may also comprise an elastic woven and/or knitted and/or worked fabric.

A webbing strap device, wherein the webbing strap has a slider loop for fixing to the first frame crossbar and/or an anchor loop or an anchor bead for fixing to the adjustment strap anchor, is particularly robust in use. The slider loop and/or the anchor loops may be formed at a respective end of the webbing strap, in particular by stitching and/or welding and/or gluing of a respective cut end of the webbing strap to a part of the webbing strap which is spaced from the respective cut end. To fix the slider loop to the first frame crossbar, the webbing strap may be passed through the first strap opening in the region of the slider loop and then be returned to a fixing point, in particular to a stitching and/or welding and/or gluing point. To fix the webbing strap to the adjustment strap anchor, the webbing strap may be guided through the adjustment strap anchor eyelet in the region of the anchor loop and then returned to a fixing point, in particular to a stitching and/or welding and/or gluing point. The webbing strap may also be attached to the adjustment strap anchor by means of an anchor bead. Preferably, the anchor bead is formed by several layers of webbing strap which are in particular laid one above the other and/or wound in the spiral form. The multiple layers of the webbing strap may be stitched together and/or joined together by substance bonding, in particular by gluing or welding. For connecting to the adjustment strap anchor, the anchor bead preferably has a bead thickness which is greater than an eyelet width of an adjustment strap anchor eyelet of the adjustment strap anchor. The eyelet width extends perpendicularly to a length extent of the adjustment strap anchor eyelet. In this way, the webbing strap may be connected to the adjustment strap anchor by form fit and hence be particularly secure.

Alternatively, the anchor bead may also be formed by a thickening body connected to the webbing strap. The bead thickness of the thickening body may be greater than the eyelet width. The thickening body may also protrude beyond the adjusting strap anchor eyelet along its length extent. The thickening body may be clamped and/or welded and/or glued and/or connected by form fit to the webbing strap.

Correspondingly to the connection of the webbing strap to the adjustment strap anchor, the webbing strap may also be connected to the first frame crossbar by means of an anchor bead in the manner described above.

A webbing strap device, wherein the webbing strap includes a thermoplastic, can be produced particularly economically and is robust in use. The webbing strap may comprise a textile material with the thermoplastic. Preferably, the thermoplastic is melted at least at one cut end of the webbing strap in order to form a melt edge. In this way, the textile webbing strap can be prevented from fraying. Preferably, the slider loop and/or the anchor loop is formed by welding a respective cut end of the webbing strap to a part of the webbing strap spaced from this cut end.

A webbing strap device, wherein the first frame crossbar, the second frame crossbar and the at least two intermediate crossbars are arranged in one crossbar plane, can be produced economically and has a high wearing comfort. The webbing strap device may be made from a plate-like semifinished product, in particular by cutting and/or material removal machining. By arranging the first frame crossbar, the second frame crossbar and the at least two intermediate crossbars in the crossbar plane, the webbing strap device has an even support surface in the region of the slider. Protruding crossbars may be avoided, which results in high wearing comfort.

A webbing strap device, wherein the slider has a positioning aid for automatable alignment and/or fixing of the slider, can be produced automatically. The positioning aid is preferably formed as a protrusion, in particular as a pin-like and/or bar-like and/or flat protrusion. The positioning aid may however also be formed as a depression. The positioning aid guarantees an alignment and/or fixing of the slider during the production process. Preferably, the positioning aid is at least partially, in particular completely covered by the webbing strap.

The positioning aid may be arranged on at least one of the frame struts and/or on the at least one intermediate crossbar and/or on the second frame crossbar.

A webbing strap device, wherein the positioning aid is arranged on the first frame crossbar, can be produced automatically. In particular, the positioning aid is arranged exclusively on the first frame crossbar. The positioning aid may form a stop for alignment and fixing of the slider.

A webbing strap device, wherein the first frame crossbar and/or the second frame crossbar and/or the at least two intermediate crossbars have a crossbar knurling to increase the friction exerted against the webbing strap, guarantees an increased friction between the webbing strap and the slider. Unintentional displacement of the webbing strap relative to the slider may thus be prevented. The crossbar knurling may be formed as a longitudinal knurling and/or as a transverse knurling.

A webbing strap device, wherein the slider has two frame struts which are each connected to the first frame crossbar, the second frame crossbar and the at least two intermediate crossbars, wherein at least one of the two frame struts has a frame knurling for easier sliding, guarantees simple movement of the slider relative to the webbing strap. Preferably, the frame knurling is arranged on an outside of the at least one frame strut. The frame knurling allows the slider to be moved relative to the webbing strap manually, in particular by hand, particularly easily.

The invention is furthermore based on the object of creating a functional device which can be produced automatically and economically.

A further desirable outcome of the present invention is to provide a functional device including a webbing strap device according to the invention, and a functional element on which the adjustment strap anchor and the adjustment loop anchor are arranged. The advantages of the functional device according to the invention correspond to the advantages of the webbing strap device already described. The functional element may optionally be connected reversibly, i.e. in particular releasably, to the adjustment strap anchor and/or the adjustment loop anchor. In this way, the webbing strap device may be exchanged. Preferably, the adjustment strap anchor and/or the adjustment loop anchor are connected to the functional element via a catch element. The connection between the webbing strap device and the functional element may thus be opened particularly easily, in particular without tools.

Preferably, the positioning aid is arranged on a side of the slider facing away from the functional element. Preferably, at least one end of the webbing strap is arranged on a side of the webbing strap device facing away from the functional element, in particular in the region of the slider loop and/or in the region of the anchor loop. In this way, a particularly high wearing comfort of the functional device is guaranteed.

The functional element may be part of a protective equipment, in particular a bodily protection, in particular protective clothing. The functional element may also be a carrier element, in particular for carrying tools and/or other utensils, in particular on the body.

A functional device, wherein the functional element includes a face protection element and/or an eye protection element and/or a respiratory protection element, can be produced particularly economically and has a high wearing comfort. Preferably, the functional element is designed to be worn on the head. A high wearing comfort of the webbing strap device is here particularly advantageous. The functional element may be configured as a respiratory protection mask and/or as protective goggles, in particular as working protective goggles or as ski goggles.

Further features, advantages and details of the invention arise the following description of an exemplary embodiment.

### BRIEF DESCRIPTION OF THE DRAWING

FIG. 1 shows a perspective depiction of a functional device according to the invention with a functional element and a webbing strap device arranged thereon,

FIG. 2 shows a diagrammatic, sectional view of the webbing strap device from FIG. 1 with an adjustment strap anchor, an adjustment loop anchor, a ladder-like slider and a webbing strap,

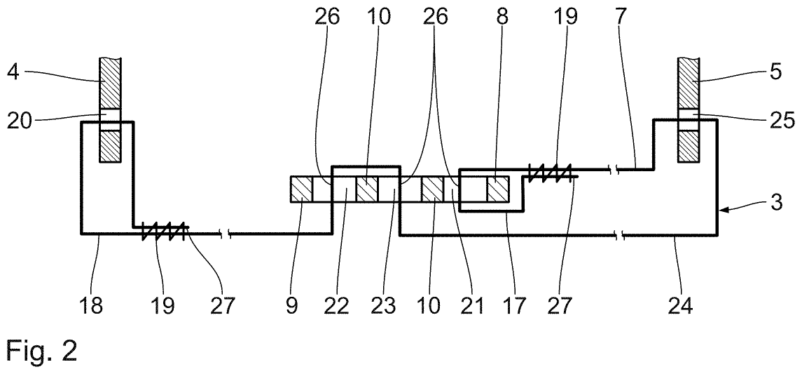

FIG. 3 shows a perspective depiction of the slider of the webbing strap device from FIG. 1, and

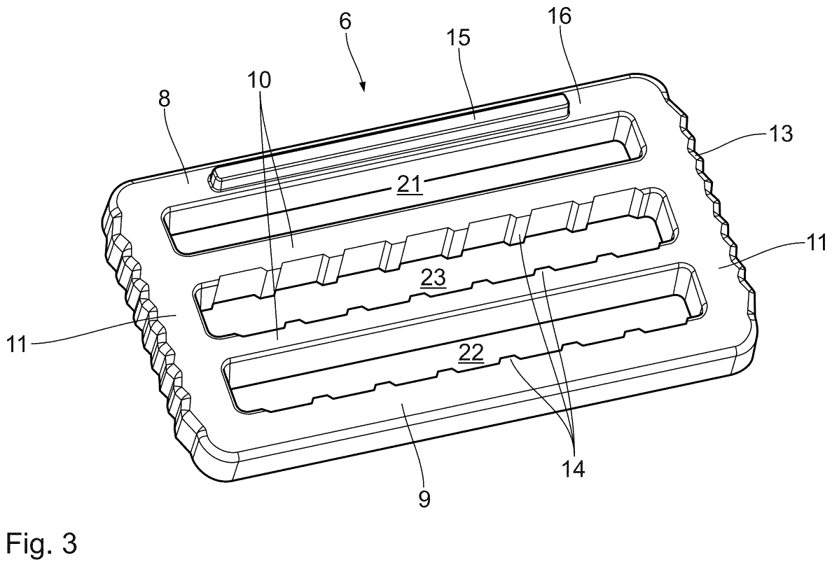

FIG. 4 shows a diagrammatic sectional view of a webbing strap device with an adjustment strap anchor, an adjustment loop anchor, a ladder-like slider and a webbing strap according to a further exemplary embodiment, wherein the webbing strap has an anchor bead for attaching to the adjustment strap anchor.

### DESCRIPTION OF THE PREFERRED EMBODIMENT

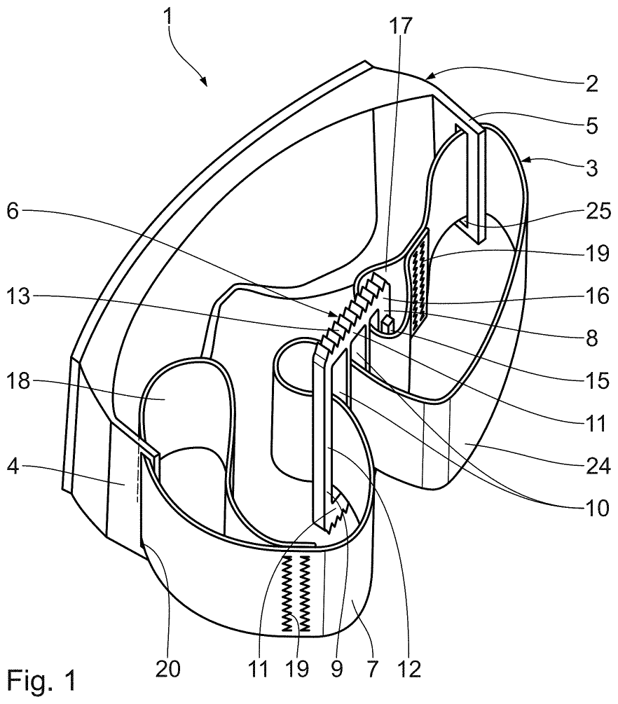

A functional device it according to FIG. 1 comprises a functional element 2 and a webbing strap device 3 arranged thereon. The functional element 2 is configured as an eye protection element. The functional device 1 is a pair of protective goggles, in particular full vision goggles.

The webbing strap device 3 comprises an adjustment strap anchor 4 and an adjustment loop anchor 5. The webbing strap device 3 is attached to the functional element 2 via the adjustment strap anchor 4 and the adjustment loop anchor 5. The adjustment strap anchor 4 and the adjustment loop anchor 5 are connected to the functional element 2 by substance bonding and hence fixedly. The webbing strap device 3 thus forms a head band for fixing the functional device 1 to the head.

The webbing strap device 3 furthermore comprises a ladder-like slider 6 and a webbing strap 7. The webbing strap 7 passes repeatedly through the slider 6, wherein the slider 6 can be moved relative to the webbing strap 7 in order to adjust a length of the webbing strap device 3 between the adjustment strap anchor 4 and the adjustment loop anchor 5.

The slider 6 comprises a first frame crossbar 8, a second frame crossbar 9 spaced from the first frame crossbar 8, and two intermediate crossbars 10 arranged between the first frame crossbar 8 and the second frame crossbar 9. The first frame crossbar 8, the second frame crossbar 9 and the two intermediate crossbars 10 are connected at both ends to a respective frame strut 11. The first frame crossbar 8, the second frame crossbar 9, the two intermediate crossbars 10 and the two frame struts 11 are arranged in a common crossbar plane 12. The two frame struts 11 together with the first frame crossbar 8 and the second frame crossbar 9 form a completely closed frame. The first frame crossbar 8, second frame crossbar 9 and the two intermediate crossbars 10 are oriented parallel to each other. The slider 6 is made from a plastic, in particular a thermoplastic.

The two frame struts each have a frame knurling 13 on an outside of the slider 6. The respective frame knurling 13 is configured such that the slider 6 can easily be moved by hand relative to the webbing strap 7.

The second frame crossbar 9 and the two intermediate crossbars 10 each have a crossbar knurling 14. The crossbar knurling 14 is arranged on a respective inside of the second frame crossbar 9 and the two intermediate crossbars 10, in particular in the direction of a geometric central point of the slider 6. The crossbar knurlings 14 are configured such that the coefficient of friction formed between the slider 6 and the webbing strap 7 is increased.

A positioning aid 15 is arranged on the first frame crossbar 8. The positioning aid 15 is formed as a bar-like protrusion and oriented parallel to the first frame crossbar 8. The positioning aid 15 is arranged on a top side 16 of the slider 6 facing away from the functional element 2.

At its first end, the webbing strap 7 has a slider loop 17, and at its second end an anchor loop 18. The slider loop 17 and the anchor loop 18 are each formed by stitching 19. The webbing strap 7 is attached to the adjustment strap anchor 4 by the anchor loop 18. For this, in the region of the anchor loop 18, the webbing strap 7 passes through an adjustment strap anchor eyelet 20 of the adjustment strap anchor 4 and back to a first stitching point.

The slider 6 has a first strap opening 21 surrounded by the first frame crossbar 8, the intermediate crossbar 10 adjacent thereto, and the two frame struts 11. The slider 6 has a second strap opening 22 framed by the second frame crossbar 9, the intermediate crossbar 10 adjacent thereto, and the two frame struts 11. The slider 6 has a middle strap opening 23 surrounded by the two intermediate crossbars 10 and the two frame struts 11. The webbing strap 7 is attached to the first frame crossbar 8 by the slider loop 17. For this, in the region of the slider loop 17, the webbing strap 7 passes through the first strap opening 21 and is guided back to a second stitching point.

The webbing strap 7 is guided through the middle strap opening 23. Between the first strap opening 21 and the middle strap opening 23, the webbing strap 7 extends in the form of an adjustment loop 24. For movable fixing of the webbing strap 7, the adjustment loop anchor 5 has an adjustment loop eyelet 25. In the region of the adjustment loop 24, the webbing strap 7 is guided through the adjustment loop eyelet 25.

Between the middle strap opening 23 and the anchor loop 18, the webbing strap 7 is guided through the second strap opening 22. The webbing strap 7 passes precisely once through each of the first strap opening 21, the second strap opening 22 and the middle strap opening 23. Adjacent passages 26 of the webbing strap 7 through the slider 6 are thus separated from each other by a respective one of the two intermediate crossbars 10. The webbing strap 7 runs round each of the two intermediate crossbars 10 on one side.

The webbing strap 7 is formed elastically. A stretch elongation of the webbing strap 7 is at least 10%. The webbing strap 7 is also formed as one piece. The webbing strap 7 is made from a textile material, preferably in a weaving process. The webbing strap 7 comprises a thermoplastic. Fraying of the webbing strap 7 is prevented by melting of the thermoplastic in the region of the two cut ends 27.

The function of the webbing strap device 3 of adjustable length, or of the functional device 1 with this webbing strap device 3, is as follows:

To attach the functional device 1 to the head, the functional element 2, in particular the eye protection element, is arranged in front of the face. The webbing strap device 3 is laid around the head, wherein it extends from the adjustment loop anchor 5, behind the head and up to the adjustment strap anchor 4. To adjust the webbing strap device 3 to the respective size of the head, the length of the webbing strap device 3 is adjustable.

In the region of the adjustment loop 24, between the slider loop 17 and the adjustment loop anchor 5, the webbing strap 7 is doubled. Between the adjustment strap anchor 4 and the second strap opening 22, the webbing strap 7 has merely a single layer. By moving the slider 6 relative to the webbing strap 7, the length of the webbing strap device 3 can be adjusted. Enlarging the adjustment loop 24 leads to a reduction in the length of the webbing strap device 3. Reducing the size of the adjustment loop 24 leads to an increase in length of the webbing strap device 3.

The slider 6 is moved relative to the webbing strap 7 manually, wherein the frame knurlings 13 facilitate the movement.

The length of the webbing strap device 3 is set such that the webbing strap 7, in particular with stretching, lies firmly against the head. The crossbar knurlings 14 guarantee an increased friction between the slider 6 and the webbing strap 7, and prevent an undesirable displacement of the slider 6 relative to the webbing strap 7.

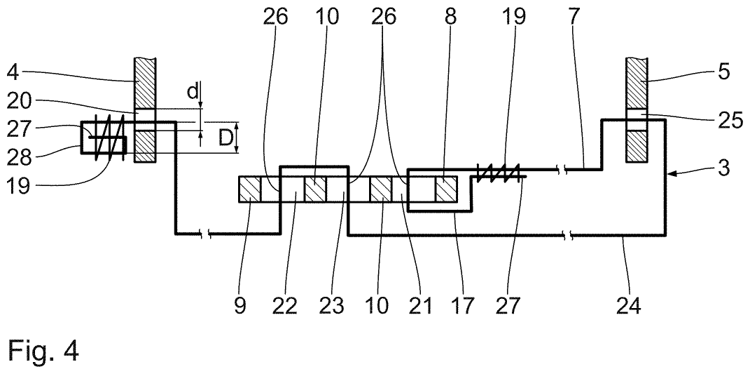

FIG. 4 shows a webbing strap device 3 according to an alternative exemplary embodiment. The webbing strap device 3 differs from the embodiment described above in that the webbing strap 7 is attached to the adjustment strap anchor 4 by means of an anchor bead 28. The anchor bead 28 comprises several layers of the webbing strap 7 laid one above the other. The multiple layers of the webbing strap 7 laid one above the other are fixedly connected together by stitching 19. For passage of the webbing strap 7, an eyelet width d of the adjustment strap anchor eyelet 20 is greater than the thickness of the webbing strap 7. A bead thickness D of the anchor bead 28 is in turn greater than the eyelet width d. This guarantees that the webbing strap 7, which is guided through the adjustment strap anchor eyelet 20, is connected by form fit to the adjustment strap anchor 4. The function of the webbing strap device 3 according to the alternative exemplary embodiment corresponds to the function of the webbing strap device 3 described above.

The design of the webbing strap device 3 according to the invention guarantees a reduction in the production cost and allows automated production. In particular because the webbing strap 7 passes only once through each of the openings of the slider 6, in particular the first strap opening 21, the second strap opening 22 and the middle strap opening 23, the one-piece webbing strap 7 can automatically be guided between the adjustment strap anchor 4, the adjustment loop anchor 5 and through the slider 6. The production process of the webbing strap device 3 thereby becomes particularly robust and economic.

1. A webbing strap device of adjustable length, comprising
an adjustment strap anchor,
an adjustment loop anchor with
an adjustment loop eyelet,
a ladder-shaped slider with
a first frame crossbar,
a second frame crossbar spaced from the first frame crossbar,
at least two intermediate crossbars arranged between the first frame crossbar and the second frame crossbar, and
at least three strap openings for guiding through of a webbing strap, and
a webbing strap attached to the first frame crossbar and to the adjustment strap anchor,
wherein the webbing strap extends through the slider between the at least two intermediate crossbars to form an adjustment loop, and
wherein in the region of the adjustment loop, the webbing strap extends through the adjustment loop eyelet,
wherein the webbing strap passes through each of the strap openings at most once and has a slider loop for fixing to the first frame crossbar.

2. The webbing strap device as claimed in claim 1, wherein the webbing strap extends through the slider adjacent to the second frame crossbar (9), between this and the at least two intermediate crossbars.

3. The webbing strap device as claimed in claim 1, wherein adjacent passages of the webbing strap through the slider are separated from each other by means of one of the at least two intermediate crossbars.

4. The webbing strap device as claimed in claim 1, wherein the webbing strap runs around the at least two intermediate crossbars only on one side.

5. The webbing strap device as claimed in claim 1, wherein the webbing strap is formed as one piece.

6. The webbing strap device as claimed in claim 1, wherein the webbing strap is formed so as to be elastic and has a stretch elongation of at least 10%.

7. The webbing strap device as claimed in claim 1, wherein the webbing strap one of the group comprising an anchor loop and an anchor bead for fixing to the adjustment strap anchor.

8. The webbing strap device as claimed in claim 1, wherein the webbing strap comprises a thermoplastic.

9. The webbing strap device as claimed in claim 1, wherein the first frame crossbar, the second frame crossbar and the at least two intermediate crossbars are arranged in one crossbar plane.

10. The webbing strap device as claimed in claim 1, the slider has a positioning aid for at least one of the group comprising automatable alignment and fixing of the slider.

11. The webbing strap device as claimed in claim 10, wherein the positioning aid is arranged on the first frame crossbar.

12. The webbing strap device as claimed in claim 1, wherein at least one of the group comprising the first frame crossbar and/or the second frame crossbar and/or the at least two intermediate crossbars have a crossbar knurling to increase the friction exerted against the webbing strap.

13. The webbing strap device as claimed claim 1, wherein the slider has two frame struts which are each connected to the first frame crossbar, the second frame crossbar and the at least two intermediate crossbars, wherein at least one of the two frame struts has a frame knurling for easier sliding.

14. A functional device, comprising
a webbing strap device as claimed in claim 1, and
a functional element on which the adjustment strap anchor and the adjustment loop anchor are arranged.

15. The functional device as claimed in claim 14, wherein the functional element comprises at least one of the group comprising a face protection element and an eye protection element and/or a respiratory protection element.
